(19) United States
(12) Patent Application Publication
(10) Pub. No.: US 2009/0326347 A1
(43) Pub. Date: Dec. 31, 2009

(54) Synchronous Light Detection Utilizing CMOS/CCD Sensors For Oximetry Sensing

(76) Inventor: Bennett Scharf, Boulder, CO (US)

Correspondence Address: NELLCOR PURITAN BENNETT LLC;ATTN: IP LEGAL, 6135 Gunbarrel Avenue, Boulder, CO, 80301, US

(21) Appl. No.: 12/165,148

(22) Filed: Jun. 30, 2008

(51) Int. Cl. A61B 5/00

(52) U.S. Cl. 600/323

(57) ABSTRACT

This disclosure describes a system and method for measuring a physiological parameter, such as a SpO₂ measurement, generated by a monitoring device having a plurality of sensors. Embodiment described herein disclose a monitoring device, such as a pulse oximeter having an array of sensor elements and an oxygen saturation module configured to calculate an estimated value of oxygen saturation of a patient's blood. This calculation is based on information received from the array of sensor elements.

### BACKGROUND

In medicine, a plethysmograph is an instrument that measures physiological parameters, such as variations in the size of an organ or body part, through an analysis of the blood passing through or present in the targeted body part, or a depiction of these variations. An oximeter is an instrument that determines the oxygen saturation of the blood. One common type of oximeter is a pulse oximeter, which determines oxygen saturation by analysis of an optically sensed plethysmograph.

A pulse oximeter is a medical device that indirectly measures the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient.

A pulse oximeter may include a light sensor that is placed at a site on a patient, usually a fingertip, toe, forehead or earlobe, or in the case of a neonate, across a foot. Light, which may be produced by a light source integrated into the pulse oximeter, containing both red and infrared wavelengths is directed onto the skin of the patient and the light that passes through the skin is detected by the sensor. The intensity of light in each wavelength is measured by the sensor over time. The graph of light intensity versus time is referred to as the photoplethysmogram (PPG) or, more commonly, simply as the “pleth.” From the waveform of the PPG, it is possible to identify the pulse rate of the patient and when each individual pulse occurs. In addition, by comparing the intensities of two wavelengths at different points in the pulse cycle, it is possible to estimate the blood oxygen saturation of hemoglobin in arterial blood. This relies on the observation that highly oxygenated blood will absorb relatively less red light and more infrared light than blood with lower oxygen saturation.

### SUMMARY

This disclosure describes a system and method for measuring a physiological parameter, such as a blood oxygen saturation measurement, using a monitoring device having a plurality of sensors. As discussed in greater detail below, the disclosure describes a monitoring device, such as a pulse oximeter, having an array of sensor elements and an oxygen saturation module configured to calculate an estimated value of oxygen saturation of a patient's blood. This calculation is based on information received from the array of sensor elements.

In another embodiment a method for measuring oxygen saturation of blood using a monitoring device having a sensor array is disclosed. According to this particular embodiment, a first sensor of the senor array is configured to measure an intensity of light of a first wavelength. A second sensor in the sensor array is configured to measure the intensity of light of a second wavelength that is different from the first wavelength. A dark reading is taken by the monitoring device in order to determine an intensity of the first wavelength and an intensity of the second wavelength in the ambient light. The first and second sensors are used to take an oximetry reading and a calculation is performed whereby the oxygen saturation is determined by subtracting an intensity of the first wavelength and an intensity of the second wavelength from the oximetry reading.

In yet another embodiment a method for negating an artifact that occurs during a photoplethysmogram is discussed in which a first measurement of an intensity of light is received at a first sensor in a sensor array, and in response to detecting the artifact, requesting a measurement of an intensity of light from a second sensor in the sensor array, the requested measurement corresponding to the first measurement.

These and various other features as well as advantages which characterize the disclosed systems and methods will be apparent from a reading of the following detailed description and a review of the associated drawings. Additional features of the systems and methods described herein are set forth in the description which follows, and in part will be apparent from the description, or may be learned by practice of the technology. The benefits and features will be realized and attained by the structure particularly pointed out in the written description and claims as well as the appended drawings.

It is to be understood that both the foregoing general description and the following detailed description are exemplary and explanatory and are intended to provide further explanation of the disclosed technology as claimed.

### BRIEF DESCRIPTION OF THE DRAWINGS

The following drawing figures, which form a part of this application, are illustrative of disclosed technology and are not meant to limit the scope of the description in any manner, which scope shall be based on the claims appended hereto.

FIG. 1 is a perspective view of a pulse oximetry system.

FIG. 2 is a block diagram of the exemplary pulse oximetry system of FIG. 1 coupled to a patient.

FIG. 3 is a block diagram of the pulse oximetry system of FIG. 2 containing an array of sensor elements.

FIG. 4 illustrates a method for measuring oxygen saturation of blood using a monitoring device having a sensor array.

### DETAILED DESCRIPTION

This disclosure describes a system and method for measuring a physiological parameter, such as a SpO₂ measurement, using a monitoring device having a plurality of sensors. As discussed in greater detail below, the disclosure describes a monitoring device, such as a pulse oximeter, having an array of sensor elements and an oxygen saturation module configured to calculate an estimated value of oxygen saturation of a patient's blood. This calculation is based on information received from the array of sensor elements.

Although the system and method described below are in the context of a pulse oximeter, it is contemplated that a monitoring device having a sensor array as described herein may be implemented by a variety of medical devices and for monitoring a variety of physiological parameters.

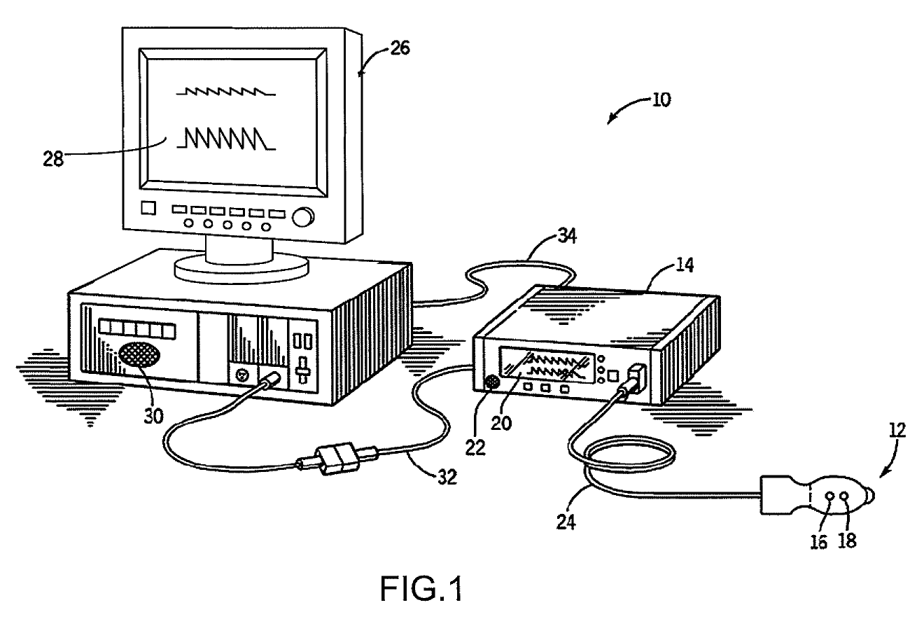

FIG. 1 is a perspective view of an embodiment of a pulse oximetry system 10. The system 10 includes a sensor 12 and a pulse oximetry monitor 14. The sensor 12 includes an emitter 16 for emitting light at two or more wavelengths into a patient's tissue. A detector 18 is also provided in the sensor 12 for detecting the light originally from the emitter 16 that emanates from the patient's tissue after passing through the tissue.

According to another embodiment and as will be described, the system 10 may include plurality of sensors forming a sensor array in lieu of the single sensor 12. Each of the sensors of the sensor array may be a complementary metal oxide semiconductor (CMOS) sensor. Alternatively, each sensor of the array may be charged coupled device (CCD) sensor. In yet another embodiment, the sensor array may be made up of a combination of CMOS and CCD sensors. The CCD sensor comprises a photoactive region and a transmission region for receiving and transmitting data while the CMOS sensor is made up of an integrated circuit having an array of pixel sensors. Each pixel has a photodetector and an active amplifier.

According to an embodiment, the emitter 16 and detector 18 may be on opposite sides of a digit such as a finger or toe, in which case the light that is emanating from the tissue has passed completely through the digit. In an embodiment, the emitter 16 and detector 18 may be arranged so that light from the emitter 16 penetrates the tissue and is reflected by the tissue into the detector 18, such as a sensor designed to obtain pulse oximetry data from a patient's forehead.

In an embodiment, the sensor or sensor array may be connected to and draw its power from the monitor 14 as shown. In another embodiment, the sensor may be wirelessly connected to the monitor 14 and include its own battery or similar power supply (not shown). The monitor 14 may be configured to calculate physiological parameters based on data received from the sensor 12 relating to light emission and detection. In an alternative embodiment, the calculations may be performed on the monitoring device itself and the result of the oximetry reading is simply passed to the monitor 14. Further, the monitor 14 includes a display 20 configured to display the physiological parameters or other information about the system. In the embodiment shown, the monitor 14 also includes a speaker 22 to provide an audible sound that may be used various other embodiments, such as for example, sounding an alarm in the event that a patient's physiological parameters are not within a predefined normal range.

In an embodiment, the sensor 12, or the sensor array, is communicatively coupled to the monitor 14 via a cable 24. However, in other embodiments a wireless transmission device (not shown) or the like may be utilized instead of or in addition to the cable 24.

In the illustrated embodiment, the pulse oximetry system 10 also includes a multi-parameter patient monitor 26. The monitor may be cathode ray tube type, a flat panel display (as shown) such as a liquid crystal display (LCD) or a plasma display, or any other type of monitor now known or later developed. The multi-parameter patient monitor 26 may be configured to calculate physiological parameters and to provide a central display 28 for information from the monitor 14 and from other medical monitoring devices or systems (not shown). For example, the multiparameter patient monitor 26 may be configured to display an estimate of a patient's blood oxygen saturation generated by the pulse oximetry monitor 14 (referred to as an “SpO₂” measurement), pulse rate information from the monitor 14 and blood pressure from a blood pressure monitor (not shown) on the display 28.

The monitor 14 may be communicatively coupled to the multi-parameter patient monitor 26 via a cable 32 or 34 coupled to a sensor input port or a digital communications port, respectively and/or may communicate wirelessly (not shown). In addition, the monitor 14 and/or the multi-parameter patient monitor 26 may be connected to a network to enable the sharing of information with servers or other workstations (not shown). The monitor 14 may be powered by a battery (not shown) or by a conventional power source such as a wall outlet.

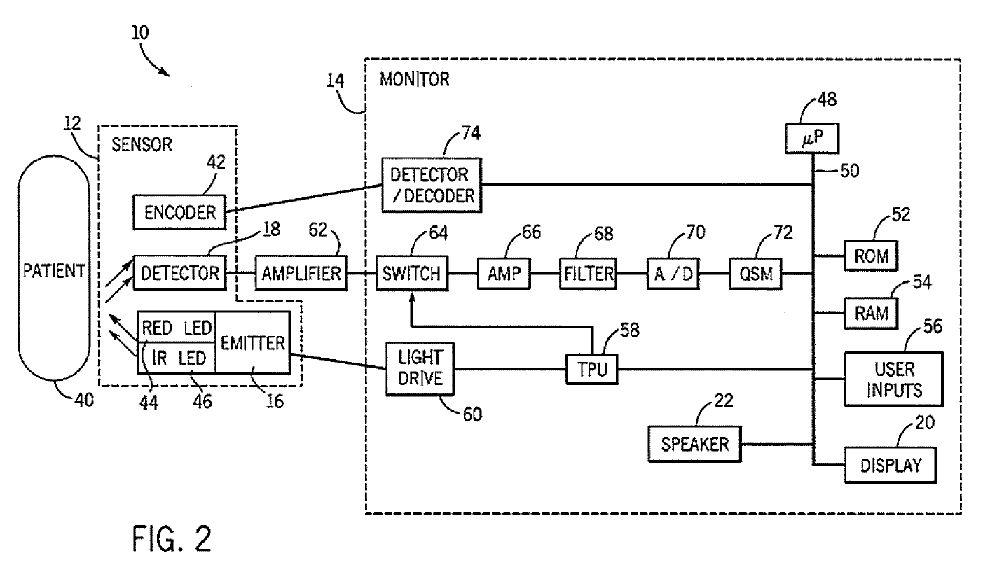

FIG. 2 is a block diagram of the embodiment of a pulse oximetry system 10 of FIG. 1 coupled to a patient 40 in accordance with present embodiments. Specifically, certain components of the sensor 12 and the monitor 14 are illustrated in FIG. 2. The sensor 12 includes the emitter 16, the detector 18, and an encoder 42. In the embodiment shown, the emitter 16 is configured to emit at least two wavelengths of light, e.g., RED and IR, into a patient's tissue 40. Hence, the emitter 16 may include a RED light emitting light source such as the RED light emitting diode (LED) 44 shown and an IR light emitting light source such as the IR LED 46 shown for emitting light into the patient's tissue 40 at the wavelengths used to calculate the patient's physiological parameters. In certain embodiments, the RED wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a RED light while a second only emits an IR light.

It should be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein light may also include any wavelength within the radio, microwave, infrared, visible, ultraviolet, or X-ray spectra, and that any suitable wavelength of electromagnetic radiation may be appropriate for use with the present techniques. Similarly, detector 18 may be chosen to be specifically sensitive to the chosen targeted energy spectrum of the emitter 16.

In an embodiment, the detector 18 may be configured to detect the intensity of light at the RED and IR wavelengths. Alternatively, each sensor in the array may be configured to detect an intensity of a single wavelength. In operation, light enters the detector 18 after passing through the patient's tissue 40. The detector 18 converts the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, the detector 18 sends the signal to the monitor 14, where physiological parameters may be calculated based on the absorption of the RED and IR wavelengths in the patient's tissue 40. An example of a device configured to perform such calculations is the Model N600x pulse oximeter available from Nellcor Puritan Bennett LLC.

In an embodiment, the encoder 42 may contain information about the sensor 12, such as what type of sensor it is (e.g., whether the sensor is intended for placement on a forehead or digit) and the wavelengths of light emitted by the emitter 16. This information may be used by the monitor 14 to select appropriate algorithms, lookup tables and/or calibration coefficients stored in the monitor 14 for calculating the patient's physiological parameters.

In addition, the encoder 42 may contain information specific to the patient 40, such as, for example, the patient's age, weight, and diagnosis. This information may allow the monitor 14 to determine patient-specific threshold ranges in which the patient's physiological parameter measurements should fall and to enable or disable additional physiological parameter algorithms. The encoder 42 may, for instance, be a coded resistor which stores values corresponding to the type of the sensor 12 or the type of each sensor in the sensor array, the wavelengths of light emitted by the emitter 16 on each sensor of the sensor array, and/or the patient's characteristics. In another embodiment, the encoder 42 may include a memory on which one or more of the following information may be stored for communication to the monitor 14: the type of the sensor 12; the wavelengths of light emitted by the emitter 16; the particular wavelength each sensor in the sensor array is monitoring; and a signal threshold for each sensor in the sensor array.

In an embodiment, signals from the detector 18 and the encoder 42 may be transmitted to the monitor 14. In the embodiment shown, the monitor 14 includes a general-purpose microprocessor 48 connected to an internal bus 50. The microprocessor 48 is adapted to execute software, which may include an operating system and one or more applications, as part of performing the functions described herein. Also connected to the bus 50 are a read-only memory (ROM) 52, a random access memory (RAM) 54, user inputs 56, the display 20, and the speaker 22.

The RAM 54 and ROM 52 are illustrated by way of example, and not limitation. Any computer-readable media may be used in the system for data storage. Computer-readable media are capable of storing information that can be interpreted by the microprocessor 48. This information may be data or may take the form of computer-executable instructions, such as software applications, that cause the microprocessor to perform certain functions and/or computer-implemented methods. Depending on the embodiment, such computer-readable media may comprise computer storage media and communication media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 provides timing control signals to a light drive circuitry 60 which controls when the emitter 16 is illuminated and multiplexed timing for the RED LED 44 and the IR LED 46. The TPU 58 also controls the gating-in of signals from detector 18 through an amplifier 62 and a switching circuit 64. These signals are sampled at the proper time, depending upon which light source is illuminated. The received signal from the detector 18 may be passed through an amplifier 66, a low pass filter 68, and an analog-to-digital converter 70. The digital data may then be stored in a queued serial module (QSM) 72 (or buffer) for later downloading to the RAM 54 as the QSM 72 fills up. In one embodiment, there may be multiple separate parallel paths having the amplifier 66, the filter 68, and the A/D converter 70 for multiple light wavelengths or spectra received.

In an embodiment, the microprocessor 48 may determine the patient's physiological parameters, such as SpO₂ and pulse rate, using various algorithms and/or look-up tables based on the value of the received signals and/or data corresponding to the light received by the detector 18. Signals corresponding to information about the patient 40, and particularly about the intensity of light emanating from a patient's tissue over time, may be transmitted from the encoder 42 to a decoder 74. These signals may include, for example, encoded information relating to patient characteristics. The decoder 74 may translate these signals to enable the microprocessor to determine the thresholds based on algorithms or look-up tables stored in the ROM 52. The user inputs 56 may be used to enter information about the patient, such as age, weight, height diagnosis, medications, treatments, and so forth. In certain embodiments, the display 20 may exhibit a list of values which may generally apply to the patient, such as, for example, age ranges or medication families, which the user may select using the user inputs 56.

The embodiments described herein relate to determining one or more statistical parameters of data from which an estimated physiological parameter value has been determined. Statistical parameters associated with the physiological parameter include parameters related to the accuracy of the estimated value such as error estimates and probability distributions of the data.

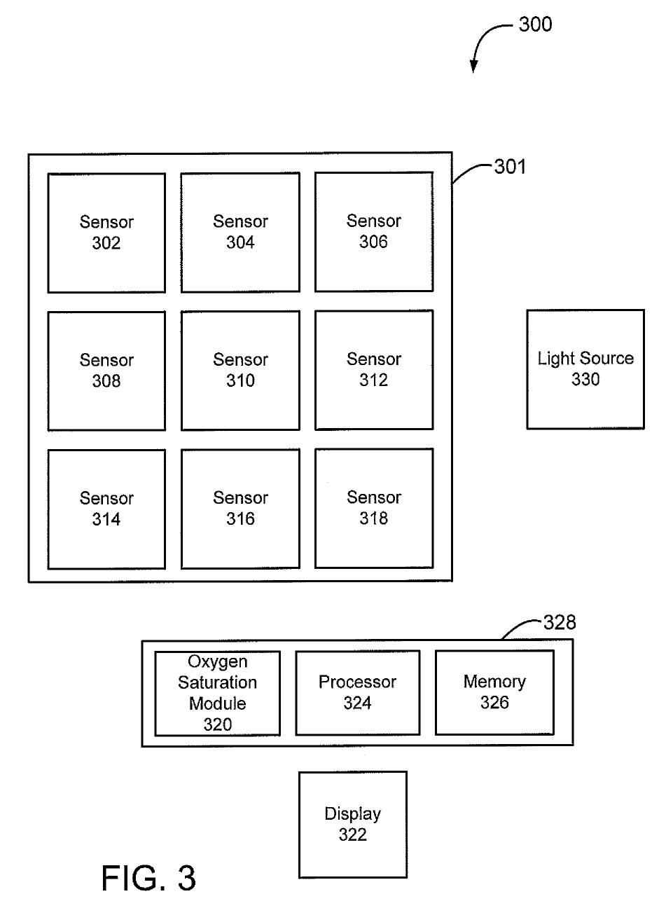

FIG. 3 is a block diagram of a pulse oximeter system 300 having a plurality of sensors forming a sensor array 301 that senses light emitted from a light source 330. As described above, the light source 330 is adapted to be positioned so that emitted light of the appropriate frequencies passes through a patient prior to being detected by one or more of the sensors of the sensor array 301. According to an embodiment, the sensors 302, 304, 306, 308, 310, 312, 314, 316, 318 of sensor array 301 are complementary metal oxide semiconductor (CMOS) sensors. Alternatively, the sensors 302, 304, 306, 308, 310, 312, 314, 316, 318 are charged coupled device (CCD) sensors. In yet another embodiment, the sensors 302, 304, 306, 308, 310, 312, 314, 316, 318 may be arranged in varying combinations of CCD and CMOS sensors. One advantage of using CMOS/CCD sensors in lieu of a single photo diode sensor as discussed above, is the way data is received and stored. In a single diode configuration the data is received as a current. In order to process and transmit the data, the data must be converted from a current to a voltage (i.e., I to V conversion). Each time a conversion is made, the quality of the signal diminishes. In contrast, both CMOS and CCD sensors receive the data as a voltage. The sensor may then sample, digitize, store or transmit the received data, all the while preserving signal quality.

The sensors 302, 304, 306, 308, 310, 312, 314, 316, 318 may be arranged in a variety of ways according to one or more embodiments. Although the sensors 302, 304, 306, 308, 310, 312, 314, 316, 318 of the sensor array 301 are shown in FIG. 3 as a 3×3 array this disclosure is not so limited. According to an embodiment, the sensor array may be an N×N array. Alternatively, the sensor array 301 may be arranged to form an M×N array. Yet additional embodiments provide that the sensors 302, 304, 306, 308, 310, 312, 314, 316, 318 may be arranged in any desired pattern such as a box, rectangle, an X, a straight line, a triangle or any combination thereof.

As the sensors may be configured in the various arrangements discussed above, the pulse oximetry system 300 is able to obtain a better signal than would normally be expected when using a pulse oximeter with a single sensor. Use of a sensor array 301 such as described herein enables an operator of the system 300 to selectively choose which received data will be processed from the one or more sensors 302, 304, 306, 308, 310, 312, 314, 316, 318 of the sensor array 301. For example, an operator may opt to use data collected from the sensors that have a signal quality over a predetermined threshold. Alternatively, the operator may choose to have the system 300 take an average of all readings obtained by each sensor 302, 304, 306, 308, 310, 312, 314, 316, 318 in the sensor array 301 in order to find the oxygenation level.

In another embodiment the use of a sensor array 301 assists in negating sensor misplacement and/or differences in skin pigmentation. For example, previous embodiments of pulse oximetry systems containing a single photo diode would not be able to obtain an accurate oxygenation reading if the sensor was misplaced or the sensor was placed on a portion of the fingertip where skin pigmentation prohibited the sensor from obtaining a strong signal. The current embodiments overcome misplacement and pigmentation problems by enabling multiple sensors to simultaneously measure light intensity at a number of different points in the array. Thus, if one or more sensors in the array have a weak signal or did not get a good reading, collected data having a stronger signal be requested from an alternate sensor in the array and/or preferentially used (e.g., in the determination of SpO₂). For example, if the sensor array 301 is arranged in an M×N format and the sensor is misplaced on a fingertip of a patient (i.e., the sensor is not placed on the center of the fingertip), a strong signal may still be obtained from a first portion of the M×N array (i.e., the portion of the array on which a majority of the finger is on). This configuration enables a strong signal to be obtained despite the operator error and the blood oxygen calculation may be determined using data obtained only from sensors having the strong signal.

In yet another embodiment, each sensor 302, 304, 306, 308, 310, 312, 314, 316, 318 of the sensor array 301 may be configured to measure an intensity of light of a different wavelength. For example, sensor 302 may be configured to measure an IR wavelength, sensor 304 may be configured to measure a Red wavelength, and sensor 306 configured to measure a Blue wavelength and so on. Other embodiments provide that a group of one or more sensors can measure a first wavelength while a second group of one or more sensors measures a second wavelength. For example, sensors 302, 304, 306 measure an IR wavelength, sensors 308, 310, 312 measure a Red wavelength and sensors 314, 316, 318 measure a Blue wavelength. It is contemplated that the sensors 302, 304, 306, 308, 310, 312, 314, 316, 318 may be configured to measure various other wavelengths and are able to be combined in a plurality of different configurations.

The above process may be accomplished through the use of filters. According to an embodiment, a filter may be coupled to each individual sensor or group of sensors in the array 301. Each filter may filter out one or more wavelengths, or alternatively ambient light, thereby allowing the sensor to measure a single wavelength.

Another advantage of using the sensor array 301 as described herein, is the ability to negate motion from readings obtained by the system 300. Inevitably, when reading the oxygenation of blood, a patient connected to the system will move their finger which causes an artifact in the data. The artifact may be negated by reading the data received at the different sensors in the sensor array. For example, in an embodiment it may be possible to eliminate errors due to the relative motion of the sensor array 301 and/or light source 330 relative to the patient due to patient movement by tracking the movement of the detected light across the different sensors of the array 301.

Once data has been received by the sensors 302, 304, 306, 308, 310, 312, 314, 316, 318 the data may be transmitted to an oxygen saturation module 320. The oxygen saturation module 320 generates a current oxygen saturation measurement from the data generated by the sensor array 301. In one embodiment the oxygen saturation module 320 may be contained in the same unit as the sensor array 301. Alternatively, the oxygen saturation module 320 may be contained in a separate housing 328. Data may be transmitted from the sensor array 301 to the oxygen saturation module 320 via a wireless connection (not shown) or via a direct cable connection (not shown). A display 322 may also be provided. In an embodiment, the display is configured to receive data directly from the sensors via wireless connection. System 300 may also include a processor 324 and a memory 326. These components may be contained in the same housing 328 as the oxygen saturation module 320.

The memory 326 may include RAM, flash memory or hard disk data storage devices. The memory stores data, which may be filtered or unfiltered data, received from the sensor array 301. The data may be decimated, compressed or otherwise modified prior to storing in the memory 326 in order to increase the time over which data may be retained.

The display 322 may be any device that is capable of generating an audible or visual notification. The display need not be integrated into the other components of the system 300 and could be a wireless device or even a monitor on a general purpose computing device (not shown) that receives data, email or other transmitted notifications from the system 300.

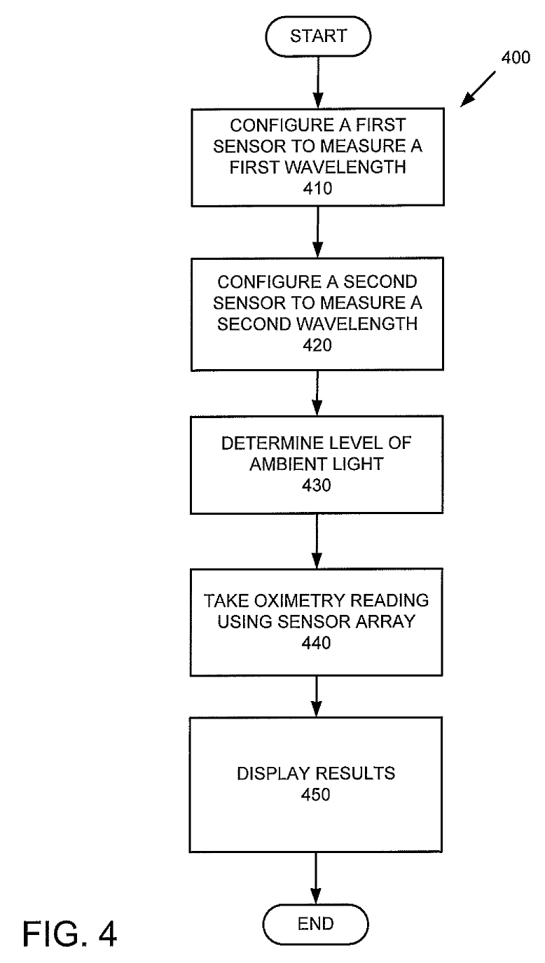

FIG. 4 is a flow chart illustrating a method 400 for measuring oxygen saturation of blood using a monitoring device having a sensor array.

According to an embodiment, step 410 provides that a first sensor of a sensor array, such as for example, sensor 302 of sensor array 301 (FIG. 3), is configured to measure an intensity of light of a first wavelength. This may be accomplished by coupling a filter to the first sensor or applying an electronic filter to the output of the sensor. For example, the filter may be adapted to filter out light of all but the first wavelength. Step 420 may be repeated for each sensor in the array selected to detect light of the first wavelength.

In step 420 a second sensor of the sensor array, such as for example, sensor 304 of sensor array 301 (FIG. 3), is configured to measure an intensity of light of a second wavelength that is different from the first wavelength. As with step 410, this may be accomplished by coupling a filter directly to the second sensor. Step 420 may be repeated for each sensor in the array selected to monitor light of the second wavelength.

In step 430 a level of ambient light in the area around the monitoring system is determined. This may be accomplished by taking a dark reading to determine the intensity of light of each wavelength in the room. Step 430 is an optional step.

In step 440 an oximetry reading is taken. In this step, the outputs of the sensors of the array are analyzed in order to obtain an SpO₂ value. An SpO₂ value may be calculated from each selected sensor independently and these values may then be averaged. In an alternative embodiment, the data from multiple sensors may be aggregated and an SpO₂ value may be calculated from the aggregate in a manner as known in the art. Many other variations are also possible.

In another embodiment, the analysis may include identifying one or more sensors providing the best data (e.g., the strongest detected intensity at the wavelengths of interest or the sensors detecting the largest waveform amplitude over time). The SpO₂ value then may be calculated from only the selected sensors. For example, in an embodiment the data for a wavelength obtained from different sensors are compared and then the best data may be selected for use in the subsequent calculation of the SpO₂ value. The comparison may be a comparison to a predetermined threshold, may be a comparison to the data from the other sensors, or a combination of the two. The data may be evaluated on intensity, signal quality, location within the array relative to the light detected by the array, detected waveform amplitude or any other suitable parameter or combination of parameters. For example, in an embodiment, only data from sensors having an intensity (or other parameter) greater than a predetermined threshold may be used to calculate the SpO₂ value.

Selecting at least one of the one or more first sensors for use in calculating the value representing the oxygen saturation

For example, in an alternative embodiment from that described in FIG. 4 in which each sensor can detect light of different wavelengths, sensor data may be selected on a wavelength-by-wavelength basis so that IR information from sensors with the best IR wavelength data may be compared to the best Red wavelength data, possibly obtained from different sensors.

Additionally, the sensor data may be analyzed spatially over of the array in order to obtain additional information that may be used to adjust the SpO₂ value or correct for errors. For example, in an embodiment the system may map the intensity of light on the array to obtain a 2-dimensional breakdown of the detected light. From this 2-dimensional data, various additional analyses may be performed such as identification of major areas of detected light through portions of the patient having high arterial blood flow. Such analysis may identify venous pulsation detection, as well as bones, arteries or other vascular elements in the patient and allow differentiation between them when calculating SpO₂ values. Further analysis may also allow the identified elements to be tracked in cases where the sensor array and/or light source moves relative to the patient.

Step 450 then displays the value representing the oxygen saturation of blood on a display.

It will be clear that the described systems and methods are well adapted to attain the ends and advantages mentioned as well as those inherent therein. Those skilled in the art will recognize that the methods and systems described within this specification may be implemented in many different manners and as such is not to be limited by the foregoing exemplified embodiments and examples. In other words, functional elements being performed by a single or multiple components, in various combinations of hardware and software, and individual functions can be distributed among software applications and even different hardware platforms. In this regard, any number of the features of the different embodiments described herein may be combined into one single embodiment and alternate embodiments having fewer than or more than all of the features herein described are possible.

While various embodiments have been described for purposes of this disclosure, various changes and modifications may be made which are well within the scope of the described technology. Numerous other changes may be made which will readily suggest themselves to those skilled in the art and which are encompassed in the spirit of the disclosure and as defined in the appended claims.

What is claimed is:

1. A pulse oximeter system comprising:
an array of sensor elements; and
an oxygen saturation module capable of calculating an estimated value of oxygen saturation of a patient's blood from information received from the array of sensor elements.

2. The pulse oximeter system of claim 1, further comprising at least one filter coupled to at least one sensor in the array of sensor elements, wherein the at least one filter filters a specified wavelength.

3. The pulse oximeter system of claim 1, wherein a first sensor in the array of sensor elements is configured to read a first wavelength and a second sensor in the array of sensor elements is configured to read a second wavelength that is different from the first wavelength.

4. The pulse oximeter system of claim 1, further comprising a wireless module coupled to the array of sensor elements, wherein the wireless module is configured to wirelessly transmit data received from the array of sensor elements to the oxygen saturation module.

5. The pulse oximeter system of claim 1, wherein the array of sensor elements is an N×N array.

6. The pulse oximeter system of claim 1, wherein the array of sensor elements is an M×N array.

7. The pulse oximeter system of claim 1, further comprising a storage module configured to store data read by each sensor in the array of sensor elements.

8. The pulse oximeter system of claim 7, wherein the oxygen saturation module is configured to take an average of the stored data from each of the sensors in the array of sensor elements taken over a time t.

9. The pulse oximeter system of claim 1, wherein the oxygen saturation module is configured to only calculate data from one or more sensors of the array of sensor elements that have a signal strength greater than a predetermined signal threshold.

10. The pulse oximeter system of claim 1, wherein the array of sensor elements are configured to detect light at multiple frequencies.

11. The pulse oximeter system of claim 1, wherein the each sensor of the array of sensor elements is a complementary metal oxide semiconductor (CMOS) sensor.

12. The pulse oximeter system of claim 1, wherein the each sensor of the array of sensor elements is a charged coupled device (CCD) sensor.

13. The pulse oximeter system of claim 1, wherein the array of sensor elements is arranged in one of a i) line; ii) a generally X configuration; iii) a generally diamond configuration; iv) a generally square configuration; and/or v) a combination thereof.

14. A method for measuring oxygen saturation of blood using a monitoring device having a sensor array, the method comprising:
configuring a plurality of first sensors in the sensor array to measure an intensity of light of a first wavelength;
configuring a plurality of second sensors in the sensor array to measure an intensity of light of a second wavelength that is different from the first wavelength; and
calculating a value representing the oxygen saturation based on data received from the first sensors and the second sensors.

15. The method of claim 14, further comprising:
comparing the data generated by the plurality of the first sensors; and
selecting at least one of the plurality of first sensors for use in calculating the value representing the oxygen saturation.

16. The method of claim 15, wherein a first sensor is selected only if the signal received at the first sensor is greater than a predetermined threshold.

17. The method of claim 14, further comprising coupling a filter to the first sensor.

18. The method of claim 15, further comprising averaging the data received from the selected at least one first sensors as part of calculating the value representing the oxygen saturation.

19. A pulse oximetry sensor comprising:
an emitter capable of emitting a plurality of wavelengths of electromagnetic radiation into a tissue;
a CMOS-type or CCD-type detector capable of receiving the plurality of wavelengths emanating from the tissue; and
a filter coupled to the detector capable of filtering substantially all wavelengths but a specified range of wavelengths, wherein the specified range of wavelengths comprises the plurality of emitted wavelengths.

20. The pulse oximetry sensor of claim 19, wherein the detector is capable of detecting electromagnetic radiation at multiple frequencies.

21. The pulse oximetry sensor of claim 19, further comprising a wireless module coupled to the detector, wherein the wireless module is configured to wirelessly transmit data received from the detectors to an oxygen saturation module.
